(19) United States
(12) Patent Application Publication
(10) Pub. No.: US 2016/0151166 A1
(43) Pub. Date: Jun. 2, 2016

(54) LOW PROFILE STANDALONE CERVICAL INTERBODY WITH SCREW LOCKING CLIPS AND METHOD OF USING SAME

(71) Applicant: Alliance Partners, LLC, San Antonio, TX (US)

(72) Inventors: Frank Morris, Austin, TX (US); Dwayne Lewis, San Antonio, TX (US); Kevin Richardson, San Antonio, TX (US)

(73) Assignee: ALLIANCE PARTNERS, LLC, San Antonio, TX (US)

(21) Appl. No.: 14/789,657

(22) Filed: Jul. 1, 2015

Related U.S. Application Data
(60) Provisional application No. 62/019,662, filed on Jul. 1, 2014.

(51) Int. Cl. A61F 2/44

(52) U.S. Cl. CPC A61F 2/442; A61F 2220/0016

(57) ABSTRACT

A vertebrae stabilization device for stabilizing adjacent vertebrae that is a cage having screw locking clips and method of using same.

### CROSS-REFERENCE TO RELATED PATENT APPLICATIONS

This application is a utility application claiming priority to U.S. Provisional Application Ser. No. 62/019,662, filed on Jul. 1, 2014, entitled “LOW PROFILE STANDALONE CERVICAL INTERBODY WITH SCREW LOCKING CLIPS,” which provisional patent application is commonly assigned to the Assignee of the present invention and is hereby incorporated herein by reference in its entirety for all purposes.

### FIELD OF THE INVENTION

Screw anchored integrated vertebral standalone cage assemblies having screw anti-backout clips.

### BACKGROUND OF THE INVENTION

In an effort to stabilize adjacent vertebrae when disks must be removed, a standalone interbody typically comprising a cage and screws may be used. The term standalone means that the cage is used without supplemental fixation, such as plates.

Screws are used which engage the cage of an interbody assembly, which screws typically tap into the vertebrae bodies above and below the cage for maintaining the cage positionally within the bodies and for providing good compression of the two vertebral bodies with the upper and lower surfaces of the cage.

To prevent screw backout, screw lockdown or anti-backout mechanisms are known in the art. These mechanisms often include some type of a member, independent of the cage, that engages both the cage and a portion of the screw when the screw is at the appropriate seated position with respect to the cage and the vertebral bodies. An audible “click” is often used by the surgeon to confirm the proper screw position. When the screws are properly positioned and engaging a backout prevention member, the screw heads and much of the screws may be within the cage body and thus hard to visually observe. The relative positions of the screw and the cage body often make difficult a visual confirmation the backout/screw/cage is properly positioned.

### SUMMARY OF THE INVENTION

A vertebrae stabilization device for stabilizing adjacent vertebrae comprising a cage having a cage body with a top surface, a bottom surface, a pair of side walls, a front wall and a rear wall, a window and a window perimeter, the front wall having a front face with multiple screw bores therethrough, a multiplicity of screws, each having a threaded shank and a head having a conical portion with a diameter greater than the shank. The screws are dimensioned to engage the screw bores and the adjacent vertebrae. A clip comprises a resilient member and at least one extension tab. The screw bores have walls dimensioned to receive and locate the clip such that the clip engages the screw head as the screw moves within the screw bore. The resilient member of the clip moves into an interference position with respect to the screw head to prevent screw backout. The tabs of the resilient member are visible from the front face of the cage and are indicative of said interference position.

A vertebrae stabilization device is provided wherein walls are dimensioned to engage a clip include walls defining a circumferential groove in a screw bore for receipt of a resilient member thereinto. Walls defining an axial groove are provided for receipt of the extension tab. The walls defining an axial groove open into the front face of the front wall. The front wall is a first color and the extension tabs are a second, contrasting color. The cage body includes a first portion comprising a first material and a second portion comprises a second material, the two portions resiliently engaging one another.

A purpose of the resilient member is to provide visual confirmation that the resilient member is in an interference position with regard to the top of the screw. In an interference position, the resilient member which also engages the cage will prevent the screw from backing out.

In general, in one aspect, the invention features a vertebrae stabilization device for stabilizing adjacent vertebrae. The vertebrae stabilization device includes a cage having a cage body with a top surface, a bottom surface, a pair of side walls, a front wall and a rear wall, and a window with a window perimeter. The front wall has a front face having a plurality of screw bores therethrough. The vertebrae stabilization device further includes a plurality of screws. Each of the screws in the plurality of screws have a screw head and are dimensioned to engage the corresponding screw bore in the plurality of screw bores. The vertebrae stabilization device further includes a plurality of clips. Each of the clips in the plurality of clips includes a resilient member and at least one extension tab. Each of the screw bores in the plurality of screw bores have walls dimensioned to receive and locate the clip in the plurality of clips that corresponds to the screw bore such that (A) the corresponding clip is operable to engage the screw head of the corresponding screw in the plurality of screws as the screw moves within the screw bore, and (B) the resilient member of the clip is operable to move into an interference position with respect to the screw head to prevent the screw from backing out.

Implementations of the inventions can include one or more of the following features:

The tabs can be visible from the front face and can be operable for indicating the interference position.

The walls of the screw bores can define a circumferential groove in the screw bore for receipt of the resilient member thereinto. The walls of the screw bore can define an axial groove for receipt of the extension tab.

The walls defining an axial groove can have an opening into the front face of the front wall.

The front wall can be a first color and the extension tabs can be a second color. The first color and the second color can be contrasting colors.

The body can include a first portion that includes a first material. The body can include a second portion that includes a second material. The first portion and the second portion can resiliently engage one another.

The first portion and the second portion can be contrasting colors.

The first portion can include a metal. The second portion can include a polymer.

The metal can include titanium. The polymer can include polyether ether ketone.

Each of the screws in the plurality of screw can have a threaded shank and the screw head can have a conical portion with a diameter greater than the threaded shank.

The resilient member of the clip can be operable to move into the interference position with respect to the screw head to prevent the screw head from backing out.

In general, in another aspect, the invention features a method of using a stabilizing device. The method includes selecting a cage having a cage body with a top surface, a bottom surface, a pair of side walls, a front wall and a rear wall, and a window with a window perimeter. The front wall has a front face having a plurality of screw bores therethrough. The plurality of screw bores each have a clip including a resilient member and at least one extension tab. The method further includes positioning the cage adjacent to a first vertebrae body and a second vertebrae body. The method further includes screwing a plurality of screws inserted through the screw bores. For each of the screws in the corresponding screw bore: (i) the screw is inserted into at least one of the first vertebrae and the second vertebrae, (ii) the screw has a screw head, (iii) the clip in the corresponding screw bore engages the screw head of the screw as the screw moves through the corresponding screw bore, and (iv) the resilient member of the clip in the corresponding screw bore moves into an interference position with respect to the screw head of the screw to prevent the screw from backing out.

Implementations of the inventions can include one or more of the following features:

The tabs can be visible from the front face and can indicate the interference position.

The walls of the screw bores can define a circumferential groove in the screw bore for receipt of the resilient member thereinto. The walls of the screw bores can define an axial groove for receipt of the extension tab.

The front wall can be a first color and the extension tabs can be a second color. The first color and the second color can be contrasting colors.

The body can include a first portion that includes a first material. The body can include a second portion that includes a second material. The first portion and the second portion can resiliently engage one another.

The first portion and the second portion can be contrasting colors.

The first portion can include a metal. The second portion can include a polymer.

Each of the screws in the plurality of screw can have a threaded shank and the screw head can have a conical portion with a diameter greater than the threaded shank.

The resilient member of the clip can be operable to move into the interference position with respect to the screw head to prevent the screw head from backing out.

### BRIEF DESCRIPTION DRAWINGS THE DRAWINGS

FIG. 1A illustrates an exploded perspective view of Applicant's interbody assembly.

FIG. 1B illustrates a side elevational view of the cage.

FIGS. 1C-1D are perspective views of the cage of the interbody assembly.

FIG. 1E is a perspective view showing the cage, screws, and insert that comprise the interbody assembly.

FIGS. 2A-2B illustrate cross-sectional views of the position of the screw with respect to the clip and the cage as the screw advances through the screw bore.

FIGS. 2C-2D are partial elevational views of the tab extensions with respect to the tab cutouts in their relative positions just prior to clip lock (FIG. 2C) and at clip lock (FIG. 2D).

FIGS. 3A-3B are perspective and side elevational views of the clip of Applicant's interbody assembly.

FIG. 4A illustrates a screw in cross-sectional view.

FIG. 4B illustrates an alternate preferred embodiment of a screw for use with Applicant's assembly.

FIG. 5 is an exploded view of one embodiment of the cage of Applicant's interbody assembly.

### REFERENCE NUMERALS

10 Interbody assembly

11 Cage

12/14/16 Clips

18 Screws

20 First, anterior portion

22 Second, posterior portion

24/26 Engagement members

24a Notched resilient arm

26a Recess/slot

28 Top surface

30 Bottom surface

32/34 Left/right side walls

36 Front wall having a front face

36a Front face

40 Rearwall

42 Window

42a Window perimeter

44/46/48 Walls of screw guide bores (left to right)

54/56/58 Clip cutouts

54a/56a/58a Tab cutouts

54b/56b/58b Clip cutouts

60 Resilient connector arm

62/64 Tab extensions

70 Head

72 Shank

74 Threads

76 Self-cutting tip

78 Conical portion

79 Bottom of head

80 Top surface of head

82 Tool engaging extension/recess

### DETAILED DESCRIPTION OF THE PREFERRED EMBODIMENT

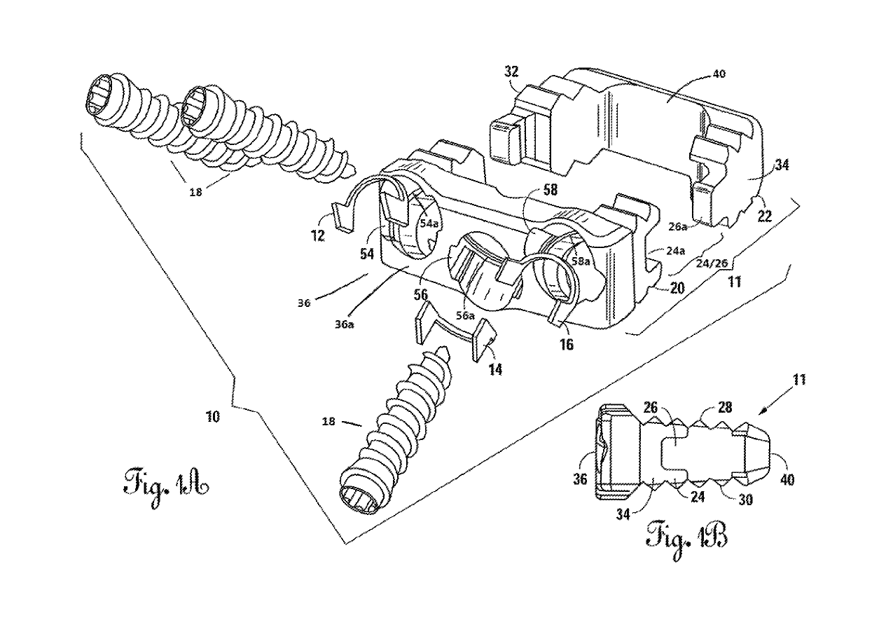

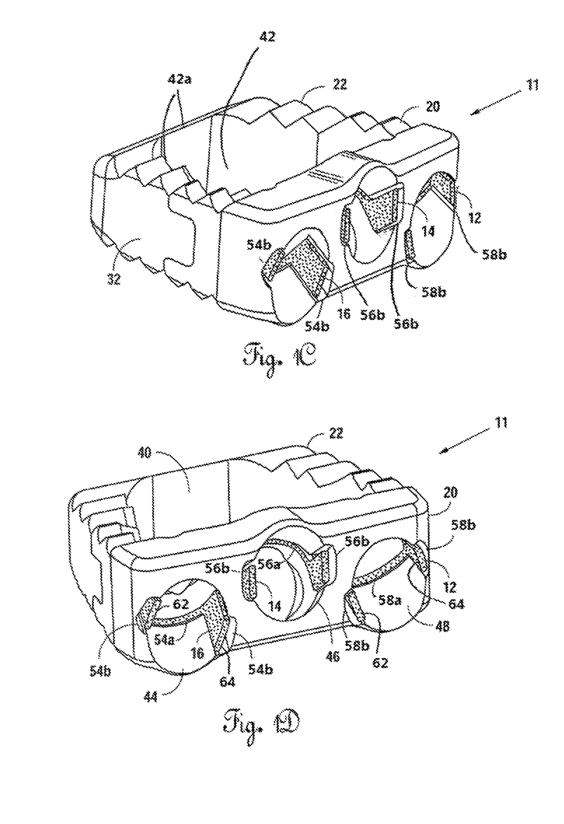

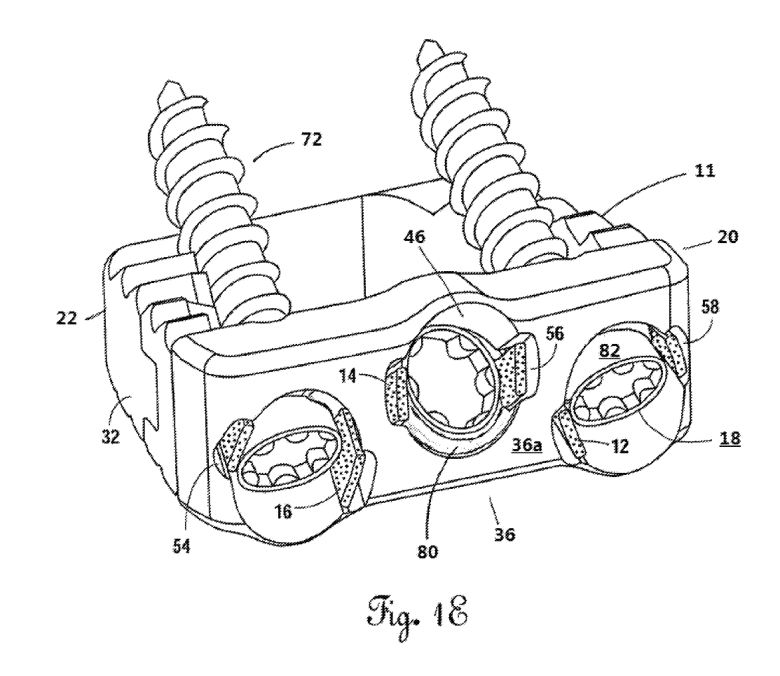

FIG. 1A illustrates the three main components of the interbody assembly 10 of the present invention. Cage 11 functions, in part, to maintain separation between a vertebra above and a vertebra below the interbody assembly. Screws 18 (a total of three as shown in the embodiment illustrated in FIG. 1A) provide fixation or location and stability of the cage to the vertebrae above and the vertebrae below the cage. Resilient clips 12/14/16, in the embodiment illustrated in FIG. 1A, engage both the cage 11 and the top of the screws 18 when the screw 18 is fully inserted into the cage 11 in a manner that prevents the screw 18 from, in time, backing out. Cages, clips, and screws generally are known in the art.

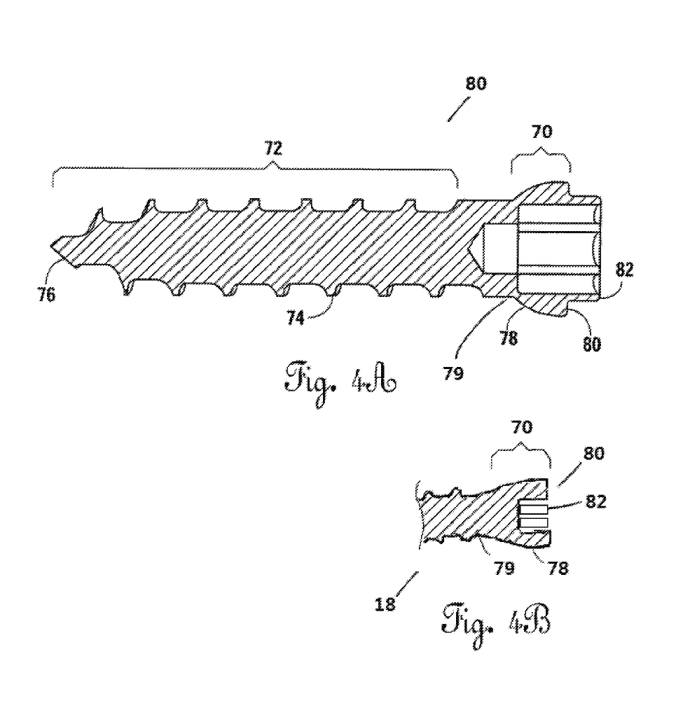

As seen in FIGS. 1A-5, the cage 11 of the present invention, in one embodiment, may include a first, anterior portion 20 and a second posterior portion 22. Engagement members 24/26 provide for engagement, such as tool-less resilient engagement, of a notched resilient arm 26a with a recess/slot 24a, which engagement couples the first portion 20 to the second portion 22 in a locking, rigid manner. In one embodiment, a first portion 20 is made of titanium or other suitable metal and a second portion 22 is made of PEEK (polyether ether ketone) or other suitable non-metallic material. A function of two parts made of dissimilar materials to form cage 11 is for support and color contrast.

As oriented in FIGS. 1A-1B, cage 11 is shown as having a top surface 28 and a bottom surface 30, the plane of the top surface 28 and bottom surface 30 angled to reflect the lordotic relationship, to match normal lordotic curvature, as is known in the art. Cage 11 is also seen to have left and right side walls 32/34. A front wall 36 engages the two side walls 32/34 and may have a front face 36a thereon. A rear wall 40 connects the rear portions of the two side walls 32/34. The side walls 32/34, front wall 36, and rear wall 40 forming a generally rectangular structure with at least one window 42 therein (shown in FIG. 1C). Window 42 is defined by a window perimeter 42a and may receive a graft material or otherwise allow bone from the upper and lower vertebrae bodies to fuse through the cage 11.

Cage 11 may include multiple walls 44/46/48 defining multiple screw guide bores (see FIG. 1D, left to right). Within each screw bore is a clip cutout 54/56/58. Clip cutouts are shaped to receive resilient clips 12/14/16 thereinto as seen in FIG. 1A.

Clip cutouts 54/56/58 may include circumferentially (typically about 180° around the screw bore) connector arm cutouts 54a/56a/58a as well as tab cutouts 54b/56b/58b. Clips 12/14/16 are resilient, and clips 12/14/16 may be squeezed slightly together at the tabs and inserted into the bore (without the screws in the bore), released adjacent each of the clip cutouts and they will pop out to engage the tab cutouts and connector arm cutouts as seen, for example, as shown in FIG. 1D. At least a portion of the bottom walls of the tabs extend into the screw bores when no screw is in bore.

Clips 12/14/16 are typically made out of a resilient, metallic material, such as titanium, in a color highly contrasting to the color of the front wall. For example, in one embodiment, the first portion 20, including front wall 36 and front face 36a, are comprised of a gold anodized titanium and the clips, including tab extensions 62/64, are green. Because of the function of visual confirmation of clip seating (interference position), it is believed the color contrast is advantageous. It is also noted that neither the color of the cage nor the clip are colors typically found in or about the operation site, that is, the colors of bone or bodily fluids.

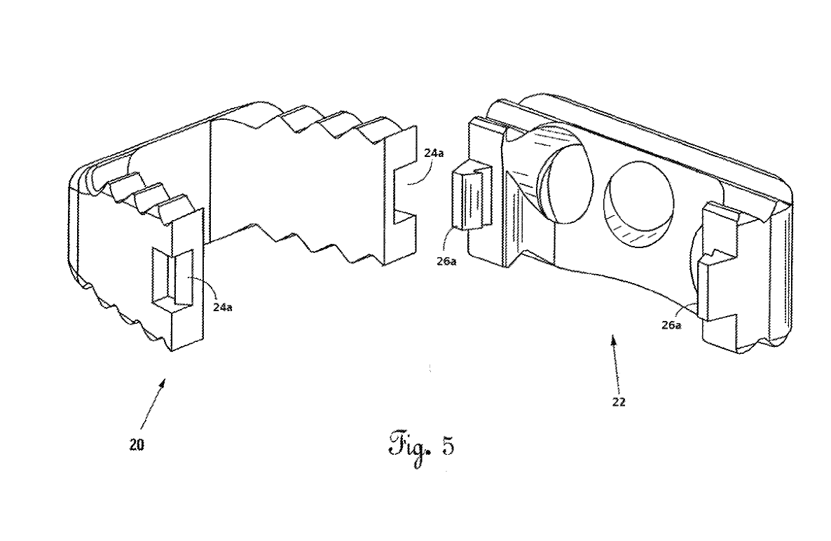

As seen in FIGS. 1A, 1E, 2A-2B, and 4A, screws 18 are generally comprised of a head 70 having a top surface 80, a threaded shank 72 having threads 74 thereon. The threads 74, in one embodiment, have a bone-cutting tip 76 through the removed thereof. A tool engaging extension (FIG. 4A) or recess (FIG. 4B) 82 may extend from or recess into the top surface of the head 70.

Head 70 is seen to include a bottom 79 and conical portion 78 having outwardly tapered (from bottom to top) sidewalls thereon. The tool engaging portion is for driving the screw through the screw guide bores and then into the bone. The screw guide bores have an axis that will guide the screws such that the outer two are directed up or down and the center is directed the other way.

When the screws 18 are inserted fully into the screw bores, the conical portion is flush against a conical portion of the removed ends of the screw bores and such that the center points of the screw heads are aligned on a line that, in one embodiment may approximate the midline of the front wall.

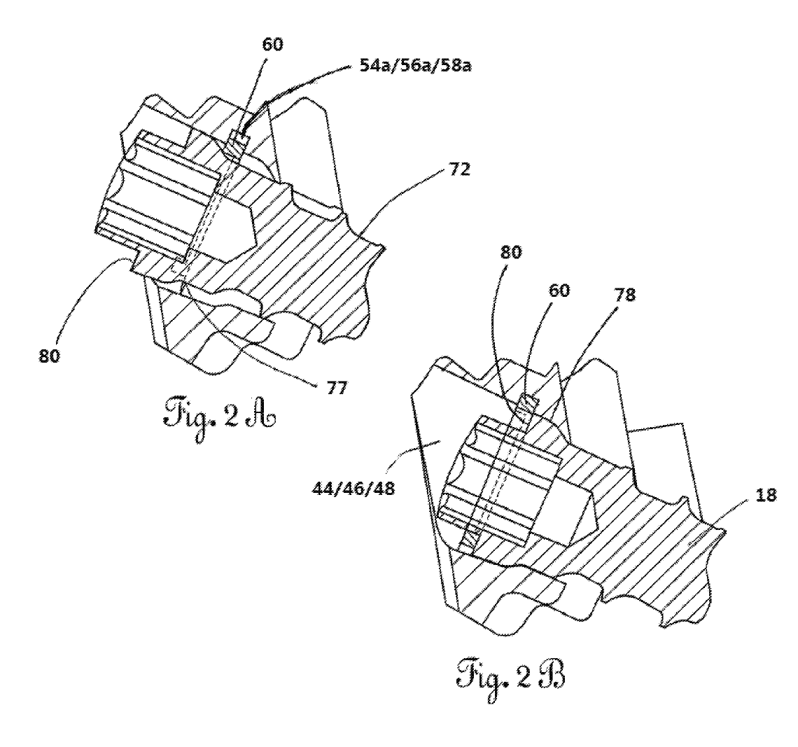

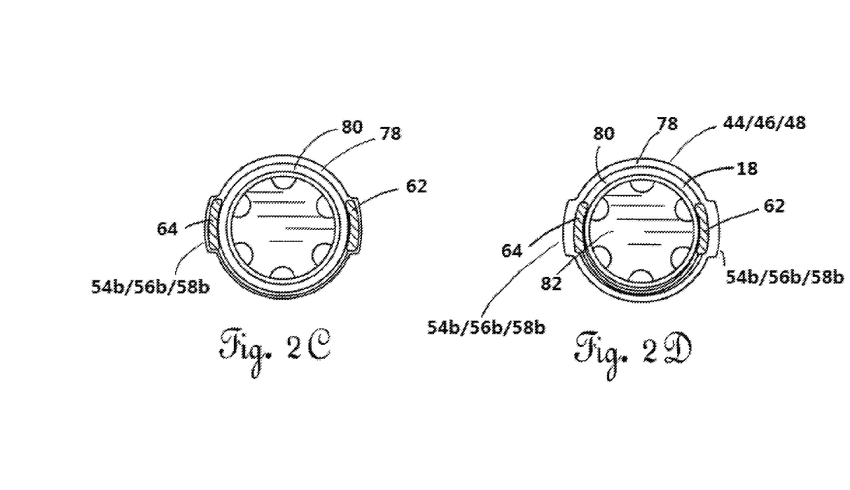

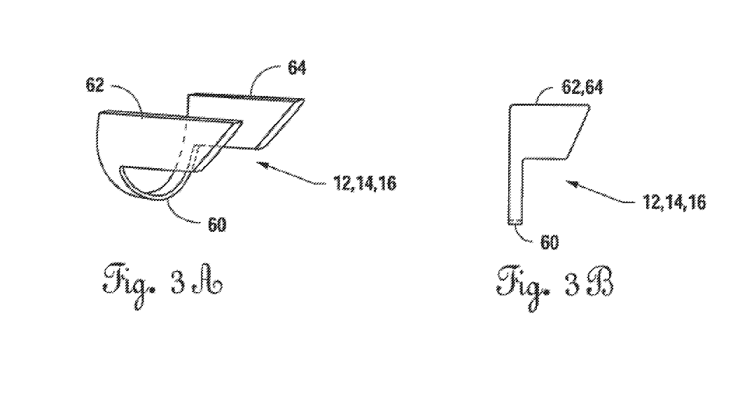

Referring to FIGS. 2A-2B, it is seen that tab cutouts 54b/56b/58b are generally configured with the same perimeter shape (but slightly larger) when viewed in the views of FIGS. 2C-2D of the tabs which they are received thereinto. Moreover, as illustrated in FIG. 2C, the tabs are pushed flush against the wall of the cutouts, and the outer face of the tabs conforms to the inner face of the cutouts. As the screw 18 advances into the screw bore, the threads of the shank cut into the bone, the removed (outer) end of the tabs will first encounter the lower end 79 of conical portion 78 of the screw head and be pushed outward so they are generally flush as illustrated in FIG. 2C. FIG. 2A shows the connector arm 60 just at its initial encounter of the lower end 70 of the screw.

At this point, the tabs are in contact with the upper area of conical portion 78. Continued advancement brings the resilient connector arms to the position shown in FIG. 2C, in which the inserts are fully seated into the cutouts and also where the connector arms 60 are fully seated in connector arm cutouts 54a/56a/58a. The instant the screw advances past that position, the resilient arms will pop or snap into a position on top of head 80 (see FIG. 1E center screw). The removed end of the tab extensions 62/64 will move from the position shown in FIG. 2C to the position shown in FIG. 2D, where it becomes spaced apart providing a visual indication to the surgeon that the clips (anti-backout) are in an interference position.

A number of embodiments of the invention have been described. Nevertheless, it will be understood that various modifications may be made without departing from the spirit and scope of the invention. Accordingly, other embodiments are within the scope of the following claims.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, other embodiments are within the scope of the following claims. The scope of protection is not limited by the description set out above, but is only limited by the claims which follow, that scope including all equivalents of the subject matter of the claims.

What is claimed is:

1. A vertebrae stabilization device for stabilizing adjacent vertebrae comprising:
(a) a cage having a cage body with a top surface, a bottom surface, a pair of side walls, a front wall and a rear wall, and a window with a window perimeter, wherein the front wall has a front face having a plurality of screw bores therethrough;
(b) a plurality of screws, wherein each of the screws in the plurality of screws have a screw head and are dimensioned to engage the corresponding screw bore in the plurality of screw bores; and
(c) a plurality of clips, wherein
(i) each of the clips in the plurality of clips comprises a resilient member and at least one extension tab, and
(ii) each of the screw bores in the plurality of screw bores have walls dimensioned to receive and locate the clip in the plurality of clips that corresponds to the screw bore such that
(A) the corresponding clip is operable to engage the screw head of the corresponding screw in the plurality of screws as the screw moves within the screw bore, and
(B) the resilient member of the clip is operable to move into an interference position with respect to the screw head to prevent the screw from backing out.

2. The vertebrae stabilization device of claim 1, wherein the tabs are visible from the front face and are operable for indicating the interference position.

3. The vertebrae stabilization device of claim 1, wherein the walls of the screw bores
(a) define a circumferential groove in the screw bore for receipt of the resilient member thereinto, and
(b) define an axial groove for receipt of the extension tab.

4. The vertebrae stabilization device of claim 3, wherein the walls defining an axial groove have an opening into the front face of the front wall.

5. The vertebrae stabilization device of claim 1, wherein
(a) the front wall is a first color and the extension tabs are a second color, and
(b) the first color and the second color are contrasting colors.

6. The vertebrae stabilization device of claim 1, wherein
(a) the body comprises a first portion comprising a first material,
(b) the body comprises a second portion comprises a second material,
(c) the first portion and the second portion resiliently engaging one another.

7. The vertebrae stabilization device of claim 6, wherein the first portion and the second portion are contrasting colors.

8. The vertebrae stabilization device of claim 6, wherein
(a) the first portion comprises a metal, and
(b) the second portion comprises a polymer.

9. The vertebrae stabilization device of claim 8, wherein
(a) the metal comprises titanium, and
(b) the polymer comprises polyether ether ketone.

10. The vertebrae stabilization device of claim 1, wherein each of the screws in the plurality of screw has a threaded shank and the screw head has a conical portion with a diameter greater than the threaded shank.

11. The vertebrae stabilization device of claim 10, wherein the resilient member of the clip is operable to move into the interference position with respect to the screw head to prevent the screw head from backing out.

12. A method of using a stabilizing device comprising the steps of:
(a) selecting a cage having a cage body with a top surface, a bottom surface, a pair of side walls, a front wall and a rear wall, and a window with a window perimeter, wherein
(i) the front wall has a front face having a plurality of screw bores therethrough,
(ii) the plurality of screw bores each have a clip comprising a resilient member and at least one extension tab;
(b) positioning the cage adjacent to a first vertebrae body and a second vertebrae body;
(c) screwing a plurality of screws inserted through the screw bores, wherein for each of the screws in the corresponding screw bore
(i) the screw is inserted into at least one of the first vertebrae and the second vertebrae,
(ii) the screw has a screw head,
(iii) the clip in the corresponding screw bore engages the screw head of the screw as the screw moves through the corresponding screw bore, and
(iv) the resilient member of the clip in the corresponding screw bore moves into an interference position with respect to the screw head of the screw to prevent the screw from backing out.

13. The method of claim 12, wherein the tabs are visible from the front face and indicate the interference position.

14. The method of claim 12, wherein the walls of the screw bores
(a) define a circumferential groove in the screw bore for receipt of the resilient member thereinto, and
(b) define an axial groove for receipt of the extension tab.

15. The method of claim 12, wherein
(a) the front wall is a first color and the extension tabs are a second color, and
(b) the first color and the second color are contrasting colors.

16. The method of claim 12, wherein
(a) the body comprises a first portion comprising a first material,
(b) the body comprises a second portion comprises a second material,
(c) the first portion and the second portion resiliently engaging one another.

16. The method of claim 16, wherein the first portion and the second portion are contrasting colors.

18. The method of claim 16, wherein
(a) the first portion comprises a metal, and
(b) the second portion comprises a polymer.

19. The method of claim 16, wherein each of the screws in the plurality of screw has a threaded shank and the screw head has a conical portion with a diameter greater than the threaded shank.

20. The method of claim 19, wherein the resilient member of the clip moves into the interference position with respect to the screw head to prevent the screw from backing out.
